(19) United States
(12) Patent Application Publication
(10) Pub. No.: US 2025/0313540 A1
(43) Pub. Date: Oct. 9, 2025

(54) PROCESS FOR THE PREPARATION OF [1,4,5]-OXADIAZEPINE DERIVATIVES

(71) Applicant: Syngenta Crop Protection AG, Basel (CH)

(72) Inventors: Linhua Wang, Greensboro, NC (US); Robert Sardzik, Stirlingshire (GB); Joseph Pike Mitchener, Jr., Greensboro, NC (US)

(73) Assignee: Syngenta Crop Protection AG, Basel (CN)

(21) Appl. No.: 18/860,397

(22) PCT Filed: Apr. 20, 2023
(86) PCT No.: PCT/EP2023/060257

§ 371 Date: Oct. 25, 2024

(30) Foreign Application Priority Data
Apr. 29, 2022 (GB) 2206290.5

(51) Int. Cl. C07D 273/06; B01D 3/36; B01J 31/02; C07D 498/04

(52) U.S. Cl. CPC C07D 273/06; B01D 3/36; B01J 31/0237; B01J 31/0268; B01J 31/0271; C07D 498/04

(57) ABSTRACT

A process for the preparation of a compound of formula (I):

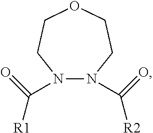

comprising the reaction of:

R¹—C(O)—NH—NH—C(O)—R², with R³—CH₂—CH₂—O—CH₂—CH₂—R⁴,
in the presence of an alcoholic solvent;
wherein R¹ and R² are independently selected from straight or branched chain alkyl groups, or are bonded to form a 4, 5, or 6-membered heterocycle, and
wherein R³ and R⁴ are independently selected from a halide or a sulphate.

The present invention relates to a novel process for the preparation of [1,4,5]-oxadiazepines derivatives and their use as intermediates in the preparation of herbicides of the tetrahydropyrazolodione type.

Pinoxaden ([8-(2,6-diethyl-4-methylphenyl)-7-oxo-1,2,4,5-tetrahydropyrazolo[1,2-d][1,4,5]oxadiazepin-9-yl]2,2-dimethylpropanoate) is a well-known graminicidal herbicide. The current commercial process for the preparation of pinoxaden involves the preparation of diacetyloxadiazepine (DAODA) as a key intermediate.

DAODA is typically prepared from the reaction of diacetylhydrazine (DAH) and dichloroethyl ether (DCEE) in the presence of dimethyl sulphoxide (DMSO). This is described in WO 03/051853 A1.

However, the use of DMSO has a significant number of technical problems associated with it. There are significant process safety issues due to the thermal decomposition of DMSO, low isolated yields, poor volume yields and a high number of waste streams. The process as a whole has a high level of complexity and cost due to the need for at least three distillation steps and difference solvents for the reaction and for crystallisation.

There is thus a need for a process that remedies the above technical problems.

The present invention therefore provides a process for the preparation of a compound of formula (I):

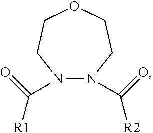

comprising the reaction of:

R¹—C(O)—NH—NH—C(O)—R², with R³—CH₂—CH₂—O—CH₂—CH₂—R⁴,
in the presence of an alcoholic solvent;
wherein R¹ and R² are independently selected from straight or branched chain alkyl groups, or are bonded to form a 4, 5, or 6-membered heterocycle, and
wherein R³ and R⁴ are independently selected from a halide or a sulphate.

The claimed process offers a significant advantage to the use of DMSO as it does not suffer from thermal instability leading to potential major accident hazard and can therefore be safely ran at a higher process concentration. It also avoids the combination of DCEE/DMSO which is highly toxic. Furthermore, the process enables the removal of a solvent swap from the process, as the process solvent may be used for both the reaction and the crystallisation of the product. Together with a reduction in inorganic and solvent waste, the process is more economical than those known in the art.

Preferably, R¹ and R² are independently selected from a straight chain alkyl groups, preferably with a chain length of from 1 to 4 carbons, even more preferably selected from methyl or ethyl. R¹ and R² may both be methyl.

In an alternative preferred embodiment, R¹ and R² are bonded to form a 5-membered heterocycle derived from pyrazolidine (i.e., R¹ and R² represent the same CH₂ moiety).

Optionally, the ring may have one or more carbonyl groups. Preferably the ring comprises two carbonyl groups to form a pyrazolidinedione derivative, even more preferably the carbonyl groups are in the 3 and 5 positions. The ring may be substituted in any fashion, for instance the ring may have a phenyl substituent (e.g., at position 4), which in itself may be substituted.

Preferably R³ and R⁴ are independently selected from methyl sulfonyl (e.g., 2-(2-methylsulfonyloxyethoxy)ethyl methanesulfonate), chlorine and/or bromine; most preferably R³ and R⁴ are chlorine (DCEE).

### Solvent System

The alcoholic solvent may be a poly- or monohydric alcohol, preferably a monohydric alcohol. Advantageously, the alcohol is a C₁₋₇ alcohol (e.g. methanol), such as a C₂₋₆ alcohol (e.g., ethanol). The alcohols may be straight or branched chain alcohols.

Most advantageously, the solvent is selected from butanol, pentanol (such as n-pentanol and/or iso-pentanol) or 2-methoxyethanol, preferably butanol (such as n-butanol).

The optimum solvent for the process described herein must:
1) be chemically compatible with the process (i.e., thermally stable);
2) have a high polarity for rate enhancement;
3) provide good solubility of the base and the DAH salt;
4) be immersible with water (which is advantageous as single solvent for reaction and crystallization); and
5) have an adequate boiling point in view of the reaction temperature of from 115 to 125° C.

It was surprisingly found that n-butanol exhibits all of the criteria (1) to (5) above.

### Phase Transfer Catalyst

Advantageously, the process is carried out in the presence of a phase-transfer catalyst (PTC) in order to increase the speed of the reaction.

Preferably the PTC is
1) a nucleophilic catalyst;
2) an ammonium catalyst; or
3) a phosphonium catalyst.

Where the PTC is a nucleophilic catalyst, it is preferably selected from 1,4-diazabicyclo[2.2.2]octane (DABCO), quinuclidine, 1,8-Diazabicyclo[5.4.0]undec-7-ene (DBU), and/or 4-Dimethylaminopyridine (DMAP).

Where the PTC is an ammonium catalyst it preferably has the formula R^{a}R^{b}R^{c}NX, where R^{a}, R^{b} and R^{c} are independently selected from alkyl, substituted alkyl and phenyl groups and X is selected from halide, hydroxide or sulphate.

More preferably it is selected from Tetramethylammonium chloride (TMAC), Tetrabutylammonium bromide (TBAB), N-Methyl-N,N,N-trioctylammonium chloride (ALIQUAT 336), Trimethylamine hydrochloride (TMA HCl) and/or 1,4,7,10,13,16-hexaoxacyclooctadecane (18-CROWN-6).

Where the PTC is a phosphonium catalyst it preferably has the structure R^{a}R^{b}R^{c}PX, where R^{a}, R^{b} and R^{c} are independently selected from alkyl, substituted alkyl and phenyl groups, and X is selected from halide, hydroxide or sulphate.

More preferably it is selected from (Methoxymethyl)triphenylphosphonium chloride (MMTPPCl), Formylmethyltriphenylphosphonium chloride (FMTPPCl), n-butyltriphenylphosphonium chloride (BuTPPCl), Benzyltriphenylphosphonium chloride (BenzylTPPCl), Tetraphenylphosphonium bromide (TPPBr), n-propyltriphenylphosphonium bromide (PrTPPBr), tetrabutylphosphonium hydroxide (TBPOH), tri(n-hexyl)tetradecylphosphonium chloride (THTDPCl), Tetra(hydroxymethyl)phosphonium chloride (THMPCl), Tetrabutylphosphonium bromide (TBPBr), Triphenylphosphine (TPP), Tributyltetradecylphosphonium chloride (TBTDPCl) and Tetrabutylphosphonium chloride (TBPCl).

The PTC is preferably 1,4-diazabicyclo[2.2.2]octane (DABCO) or a phosphonium catalyst, even more preferably 1,4-diazabicyclo[2.2.2]octane (DABCO) or a phosphonium catalyst selected from tributyltetradecylphosphonium chloride (TBTDPCl), tetrabutylphosphonium chloride (TBPCl) and/or tetrabutylphosphonium hydroxide (TBPOH).

These catalysts have been found to improve both rate and yield, as well as being stable at the relevant reaction temperatures.

Advantageously the PTC is present in an amount of from 0.005 to 0.05 molar equivalent relative to R¹—C(O)—NH—NH—C(O)—R², preferably from 0.01 to 0.02 molar equivalents.

### Base System

It has been found that the presence and identity of a base plays a significant role in the optimisation of the process. Thus, advantageously, the process is carried out in the presence of a base. The base may be selected from potassium carbonate, sodium carbonate and caesium carbonate, but is preferably potassium carbonate.

Preferably the base is present in an amount of from 1.5 to 3.0 molar equivalents relative to R¹—C(O)—NH—NH—C(O)—R², preferably from 1.8 to 2.5 molar equivalents.

However, the process typically requires a high loading of base, which limits yields and produces a significant amount of solid waste. The process is therefore preferably carried out in the presence of a base and a co-base in order to address these issues. The co-base may be selected from potassium hydroxide, potassium n-butoxide, sodium hydroxide and caesium hydroxide, preferably potassium hydroxide.

Advantageously, the ratio of base to co-base is from 10:1 to 1:2, preferably from 5:1 to 1:1.

### Process Stages

The process comprises a reaction stage (1) as defined herein. The reaction rate and yield are highly dependent on the pot temperature. Advantageously, the reaction temperature can be controlled by running the reaction under reflux at one atmosphere pressure or under reduced pressure. The reaction is preferably carried out at the solvent reflux temperature, preferably from 110 to 125° C.

Advantageously, the process comprises a salt filtration stage (2) subsequent to the reaction stage, preferably comprising the addition of further alcoholic solvent as defined herein. This stage may also comprise the removal of solid waste.

Preferably, there is a solvent distillation stage (3), where solvent can be recycled back to the reaction stage (1). Advantageously, but not necessarily, this can be the sole solvent distillation stage.

Alternatively, there is a second distillation stage after product filtration to recover residual solvent and excess R³—CH₂—CH₂—O—CH₂—CH₂—R⁴. The mother liquor contains the PTC and a significant amount of the reagents. The mother liquor can therefore replace part of solvent in the next reaction batch leading to an increased yield and reduced usage of reagents.

The process advantageously comprises a product crystallization stage (4). Stages (3) and (4) are preferably combined as a single stage.

Preferably, there is a filtration and drying (5) stage to isolate the compound of formula (I), advantageously comprising the addition of alcoholic solvent as defined herein to wash the product.

The reaction is sensitive to the water content and so the process preferably comprises the removal of water via azeotropic distillation. The water content can be controlled by azeotropic distillation, this has been found to be particularly effective when a co-base (such as potassium hydroxide) is used.

In a second aspect of the invention there is provided a process for preparing pinoxaden comprising a process as defined herein. Such a process may comprise the hydrolysis of a compound of formula (I) to form oxadiazepine (ODA). The ODA may be reacted with 2-(2,6-Diethyl-4-methylphenyl) malonamide in order to form a pinoxaden intermediate.

In a third aspect of the invention there is provided a compound of formula (I) produced by a process as defined in herein. Such a compound is preferably used in a process for the preparation of pinoxaden.

Unless otherwise stated all percentages are given as percentages by total weight and all embodiments and preferred features may be combined in any combination.

The invention is described by the following non-limiting Examples.

### EXAMPLES

### Example 1

TABLE 1

| | Weight | Purity | MW | Moles | Mole |
|---|---|---|---|---|---|
| Materials | (g) | (%) | (g/mol) | (moles) | Ratio |
| Diacetylhydrazine (DAH) | 40 | 98 | 116.1 | 0.338 | 1 |
| n-Butanol | 150.1 | >99.5 | 74.1 | 2.026 | 6.00 |
| Potassium carbonate | 28.3 | >99.0 | 138.2 | 0.203 | 0.60 |
| (K₂CO₃) | | | | | |
| 1,4- | 0.74 | 97 | 112.2 | 0.006 | 0.02 |
| diazabicyclo[2.2.2]octane | | | | | |
| (DABCO) (PTC) | | | | | |
| 14.5% w/w KOH in n- | 26.1 | 14.5 | 56.1 | 0.068 | 0.20 |
| Butanol (pre-charge) | | | | | |
| Dichlorodiethyl ether | 73.2 | >99.0 | 143 | 0.507 | 1.50 |
| (DCEE) | | | | | |
| 14.5 w/w % KOH in n- | 130.6 | 14.50 | 56.1 | 0.338 | 1.00 |
| Butanol (co-feed) | | | | | |

### Procedure

DAH (40 g, 1 eq), n-Butanol (150.1 g), powdered K₂CO₃ (28.3 g, 0.6 eq), and 1,4-diazabicyclo[2.2.2]octane (0.74 g) were charged to an Easymax 402 reactor (500 mL) equipped with overhead stirring, thermocouple, and reflux condenser attached to a Dean-Stark trap. A solution of 14.5 w/w % KOH in n-butanol (26 g) was fed sub-surface over 15 minutes at room temperature. The suspension was heated to gentle reflux (over 60 minutes) with distillate collected. DCEE (73.2 g, 1.5 eq) and a solution of 14.5 w/w % KOH in n-butanol (130.6 g) were fed sub-surface separately via syringe pumps. DCEE and KOH solution were both fed over 4 hours. The rate of water/solvent removal was adjusted to match the feed rate of base. The reaction was sampled periodically over a 12-hour period.

After reaction completion, the inorganic solids (58.5 g) were removed via filtration at room temperature, which was followed by n-butanol wash (110 g). The combined filtrate and wash (366.2 g) gave a solution yield of 55.3%. Concentration under reduced pressure at 55-60° C. rendered a concentrate (98.3 g) containing about 37% of DAODA.

To crystallize DAODA, the concentrate was cooled slowly (over 60 min) to −10° C. with mixing. Product isolation through filtration, a cold n-butanol wash (12 g) at −5° C., and drying under vacuum at 60° C. afforded DAODA (29.0 g) in a 46% yield with a purity of 99.1%.

### Example 2

TABLE 2

| | Weight | Purity | MW | Moles | Mole |
|---|---|---|---|---|---|
| Materials | (g) | (%) | (g/mol) | (moles) | Ratio |
| Diacetylhydrazine (DAH) | 40 | 98 | 116.1 | 0.338 | 1 |
| n-Butanol | 112.9 | >99.5 | 74.1 | 1.519 | 4.5 |
| Potassium carbonate | 28.3 | >99.0 | 138.2 | 0.203 | 0.60 |
| (K₂CO₃) | | | | | |
| 1,4- | 0.74 | 97 | 112.2 | 0.006 | 0.02 |
| diazabicyclo[2.2.2]octane | | | | | |
| (DABCO) (PTC) | | | | | |
| 14.5% w/w KOH in n- | 26.1 | 14.5 | 56.1 | 0.068 | 0.20 |
| Butanol (pre-charge) | | | | | |
| Dichlorodiethyl ether | 73.2 | >99.0 | 143 | 0.507 | 1.50 |
| (DCEE) | | | | | |
| 14.5 w/w % KOH in n- | 130.6 | 14.50 | 56.1 | 0.338 | 1.00 |
| Butanol (co-feed) | | | | | |

### Procedure

DAH (40 g, 1 eq), n-Butanol (112.9 g), powdered K₂CO₃ (28.3 g, 0.6 eq), and 1,4-diazabicyclo[2.2.2]octane (0.74 g) were charged to an Easymax 402 reactor (500 mL) equipped with overhead stirring, thermocouple, and reflux condenser attached to a Dean-Stark trap. A solution of 14.5 w/w % KOH in n-butanol (26 g) was fed sub-surface over 15 minutes at room temperature. The suspension was heated to gentle reflux (over 60 minutes) with distillate collected. DCEE (73.2 g, 1.5 eq) and a solution of 14.5 w/w % KOH in n-butanol (130.6 g) were fed sub-surface separately via syringe pumps. DCEE and KOH solution were both fed over 4 hours. The rate of water/solvent removal was adjusted to match the feed rate of base. The reaction was sampled periodically over a 12-hour period.

After reaction completion, the inorganic solids (59.2 g) were removed via filtration at room temperature, which was followed by n-butanol wash (112 g). The combined filtrate and wash (342 g) gave a solution yield of 54.7%. Concentration under reduced pressure at 55-60° C. rendered a concentrate (88.5 g) containing about 37% of DAODA.

To crystallize DAODA, the concentrate was cooled slowly (over 60 min) to −10° C. with mixing. Product isolation through filtration, a cold n-butanol wash (12 g) at −5° C., and drying under vacuum at 60° C. afforded DAODA (28.5 g) in a 45% yield with a purity of 98.6%.

### Example 3

TABLE 3

| | Weight | Purity | MW | Moles | Mole |
|---|---|---|---|---|---|
| Materials | (g) | (%) | (g/mol) | (moles) | Ratio |
| Diacetylhydrazine (DAH) | 30 | 98 | 116.1 | 0.253 | 1 |
| n-Butanol | 140.7 | >99.5 | 74.1 | 1.899 | 7.5 |
| Potassium carbonate | 28.3 | >99.0 | 138.2 | 0.203 | 0.80 |
| (K₂CO₃) | | | | | |
| Trimethylamine | 1.19 | 97 | 95.57 | 0.012 | 0.048 |
| hydrochloride (TMA HCl) | | | | | |
| (PTC) | | | | | |
| 14.5% w/w KOH in n- | 19.6 | 14.5 | 56.1 | 0.051 | 0.20 |
| Butanol (pre-charge) | | | | | |
| Dichlorodiethyl ether | 54.9 | >99.0 | 143 | 0.380 | 1.50 |
| (DCEE) | | | | | |
| 14.5 w/w % KOH in n- | 78.4 | 14.50 | 56.1 | 0.203 | 0.8 |
| Butanol (co-feed) | | | | | |

### Procedure

DAH (30 g, 1 eq), n-Butanol (140.7 g), and powdered K₂CO₃ (28.3 g, 0.8 eq were charged to an Easymax 402 reactor (500 mL) equipped with overhead stirring, thermocouple, and reflux condenser attached to a Dean-Stark trap. A solution of 14.5 w/w % KOH in n-butanol (19.6 g) was fed sub-surface over 15 minutes at room temperature. The suspension was heated to gentle reflux (over 60 minutes) with distillate collected. Trimethylamine hydrochloride (1.19 g) in 1.0 ml of water was charged. DCEE (54.9 g, 1.5 eq) and a solution of 14.5 w/w % KOH in n-butanol (78.6 g) were fed sub-surface separately via syringe pumps. DCEE and KOH solution were both fed over 4 hours. The rate of water/solvent removal was adjusted to match the feed rate of base. The reaction was sampled periodically over a 12-hour period.

After reaction completion, the inorganic solids (48.0 g) were removed via filtration at room temperature, which was followed by n-butanol wash (110 g). The combined filtrate and wash (312.2 g) gave a solution yield of 51.0%.

### Example 4

TABLE 4

| | Weight | Purity | MW | Moles | Mole |
|---|---|---|---|---|---|
| Materials | (g) | (%) | (g/mol) | (moles) | Ratio |
| Diacetylhydrazine (DAH) | 30 | 98 | 116.1 | 0.253 | 1 |
| n-Butanol | 140.7 | >99.5 | 74.1 | 1.899 | 7.5 |
| Potassium carbonate | 28.3 | >99.0 | 138.2 | 0.203 | 0.80 |
| (K₂CO₃) | | | | | |
| quinuclidine (PTC) | 0.55 | 97 | 111.2 | 0.004 | 0.019 |
| 14.5% w/w KOH in n- | 19.6 | 14.5 | 56.1 | 0.051 | 0.20 |
| Butanol (pre-charge) | | | | | |
| Dichlorodiethyl ether | 54.9 | >99.0 | 143 | 0.380 | 1.50 |
| (DCEE) | | | | | |
| 14.5 w/w % KOH in n- | 78.4 | 14.50 | 56.1 | 0.203 | 0.8 |
| Butanol (co-feed) | | | | | |

### Procedure

DAH (30 g, 1 eq), n-Butanol (140.7 g), powdered K₂CO₃ (28.3 g, 0.8 eq), and quinuclidine (0.55 g) were charged to an Easymax 402 reactor (500 mL) equipped with overhead stirring, thermocouple, and reflux condenser attached to a Dean-Stark trap. A solution of 14.5 w/w % KOH in n-butanol (19.6 g) was fed sub-surface over 15 minutes at room temperature. The suspension was heated to gentle reflux (over 60 minutes) with distillate collected. DCEE (54.9 g, 1.5 eq) and a solution of 14.5 w/w % KOH in n-butanol (78.4 g) were fed sub-surface separately via syringe pumps. DCEE and KOH solution were both fed over 4 hours. The rate of water/solvent removal was adjusted to match the feed rate of base. The reaction was sampled periodically over a 12-hour period.

After reaction completion, the inorganic solids (50.0 g) were removed via filtration at room temperature, which was followed by n-butanol wash (100 g). The combined filtrate and wash (314.4 g) gave a solution yield of 53.7%.

### Example 5

TABLE 5

| | Weight | Purity | MW | Moles | Mole |
|---|---|---|---|---|---|
| Materials | (g) | (%) | (g/mol) | (moles) | Ratio |
| Diacetylhydrazine (DAH) | 30 | 98 | 116.1 | 0.253 | 1 |
| n-Butanol | 140.7 | >99.5 | 74.1 | 1.899 | 7.5 |
| Potassium carbonate | 28.3 | >99.0 | 138.2 | 0.203 | 0.80 |
| (K₂CO₃) | | | | | |
| Tetrabutylphosphonium | 1.22 | 97 | 294.9 | 0.004 | 0.016 |
| chloride (TBPCl) (PTC) | | | | | |
| 14.5% w/w KOH in n- | 19.6 | 14.5 | 56.1 | 0.051 | 0.20 |
| Butanol (pre-charge) | | | | | |
| Dichlorodiethyl ether | 54.9 | >99.0 | 143 | 0.380 | 1.50 |
| (DCEE) | | | | | |
| 14.5 w/w % KOH in n- | 78.4 | 14.50 | 56.1 | 0.203 | 0.8 |
| Butanol (co-feed) | | | | | |

### Procedure

DAH (30 g, 1 eq), n-Butanol (140.7 g), powdered K₂CO₃ (28.3 g, 0.8 eq), and tetrabutylphosphonium chloride (1.22 g) were charged to an Easymax 402 reactor (500 mL) equipped with overhead stirring, thermocouple, and reflux condenser attached to a Dean-Stark trap. A solution of 14.5 w/w % KOH in n-butanol (19.6 g) was fed sub-surface over 15 minutes at room temperature. The suspension was heated to gentle reflux (over 60 minutes) with distillate collected. DCEE (54.9 g, 1.5 eq) and a solution of 14.5 w/w % KOH in n-butanol (78.4 g) were fed sub-surface separately via syringe pumps. DCEE and KOH solution were both fed over 4 hours. The rate of water/solvent removal was adjusted to match the feed rate of base. The reaction was sampled periodically over a 12-hour period.

After reaction completion, the inorganic solids (50.0 g) were removed via filtration at room temperature, which was followed by n-butanol wash (100 g). The combined filtrate and wash (292.8 g) gave a solution yield of 55.5%. Concentration under reduced pressure at 55-60° C. rendered a concentrate (55.8 g) containing about 43.5% of DAODA.

To crystallize DAODA, the concentrate was cooled slowly (over 60 min) to −10° C. with mixing. Product isolation through filtration, a cold n-butanol wash (12 g) at −5° C., and drying under vacuum at 60° C. afforded DAODA (20.6 g) in a 45% yield with a purity of 99%.

### Example 6

TABLE 6

| | Weight | Purity | MW | Moles | Mole |
|---|---|---|---|---|---|
| Materials | (g) | (%) | (g/mol) | (moles) | Ratio |
| Diacetylhydrazine (DAH) | 12.0 | 98 | 116.1 | 0.1013 | 1 |
| n-Butanol | 56.3 | >99.5 | 74.1 | 0.7597 | 7.50 |
| Potassium carbonate | 11.4 | >99.0 | 138.2 | 0.0801 | 0.80 |
| (K₂CO₃) | | | | | |
| Tetrabutylphosphonium | 0.6 | 98 | 294.9 | 0.0020 | 0.02 |
| chloride (PTC) | | | | | |
| 14.5% w/w KOH in n- | 7.9 | 14.5 | 56.1 | 0.0243 | 0.20 |
| Butanol (pre-charge) | | | | | |
| Dichlorodiethyl ether | 24.9 | >99.0 | 143 | 0.1722 | 1.70 |
| (DCEE) | | | | | |
| 14.5 w/w % KOH in n- | 31.4 | 14.50 | 56.1 | 0.0972 | 0.80 |
| Butanol (co-feed) | | | | | |

### Procedure

DAH (12 g 1 eq), n-butanol (56.3 g), powdered K₂CO₃ (11.4 g, 0.8 eq), and tetrabutylphosphonium chloride (0.6 g) were charged to an Easymax 102 reactor (150 mL) equipped with overhead stirring, thermocouple, and reflux condenser attached to a Dean-Stark trap. A solution of 14.5 w/w % KOH in n-butanol (7.9 g) was fed sub-surface over 15 minutes at room temperature. The suspension was heated to gentle reflux over 30 minutes. DCEE (24.9 g, 1.7 eq) and a solution of 14.5 w/w % KOH in n-butanol (31.4 g) were fed sub-surface separately via syringe pumps. DCEE was fed over 4 hours and KOH solution fed over 6 hours. The rate of water/solvent removal was adjusted to match the feed rate of base. The reaction was sampled periodically over a 16-hour period.

After reaction completion, the inorganic solids (25.4 g) were removed via filtration at room temperature followed by a n-butanol wash (30 g) at room temperature. The combined filtrate and n-butanol wash (163.5 g) gave a solution yield of 60.4%.

### Example 7

TABLE 7

| | Weight | Purity | MW | Moles | Mole |
|---|---|---|---|---|---|
| Materials | (g) | (%) | (g/mol) | (moles) | Ratio |
| Diacetylhydrazine (DAH) | 39.6 | 98 | 116.1 | 0.334 | 1 |
| n-Butanol | 186.7 | >99.5 | 74.1 | 0.7597 | 7.50 |
| Potassium carbonate | 37.1 | >99.0 | 138.2 | 0.269 | 0.80 |
| (K₂CO₃) | | | | | |
| Tetrabutylphosphonium | 1.98 | 98 | 294.9 | 0.0020 | 0.02 |
| chloride (PTC) | | | | | |
| 14.5% w/w KOH in n- | 26 | 14.5 | 56.1 | 0.067 | 0.20 |
| Butanol (pre-charge) | | | | | |
| Dichlorodiethyl ether | 82.5 | >99.0 | 143 | 0.571 | 1.70 |
| (DCEE) | | | | | |
| 14.5 w/w % KOH in n- | 103.9 | 14.50 | 56.1 | 0.2688 | 0.80 |
| Butanol (co-feed) | | | | | |

### Procedure

DAH (39.6 g, 1 eq), n-Butanol (186.7 g), powdered K₂CO₃ (37.1 g, 0.8 eq), and tetrabutylphosphonium chloride (1.98 g) were charged to an Easymax 402 reactor (500 mL) equipped with overhead stirring, thermocouple, and reflux condenser attached to a Dean-Stark trap. A solution of 14.5 w/w % KOH in n-butanol (26 g) was fed sub-surface over 30 minutes at room temperature. The suspension was heated to gentle reflux (over 30 minutes). DCEE (82.5 g, 1.7 eq) and a solution of 14.5 w/w % KOH in n-butanol (103.9 g) were fed sub-surface separately via syringe pumps. DCEE was fed over 4 hours and KOH solution fed over 6 hours. The rate of water/solvent removal was adjusted to match the feed rate of base. The reaction was sampled periodically over a 12-hour period. The GC chemical yield for DAODA was 55.3%.

After reaction completion, the inorganic solids (57.4 g) were removed via filtration at room temperature, which was followed by n-butanol wash (112 g). The combined filtrate (311.9 g) and wash (112 g) were concentrated under reduced pressure at 55-60° C. to render a concentrate (101.5 g) containing about 35% of DAODA.

To crystallize DAODA, the concentrate was cooled slowly (over 60 min) to −10° C. with mixing. Product isolation through filtration, a cold n-butanol wash (20 g) at −5° C., and drying under vacuum at 60° C. afforded DAODA (30.0 g) in a 48.7% yield with a purity of 99%. The mother liquor (64 g) containing DAODA (5.1 g, 8.2% yield) and n-butanol wash (18.5 g) containing DAODA (0.7 g, 0.9% yield) were main sources of product loss.

### Example 8

TABLE 8

| | Weight | Purity | MW | Moles | Mole |
|---|---|---|---|---|---|
| Materials | (g) | (%) | (g/mol) | (moles) | Ratio |
| Diacetylhydrazine (DAH) | 12.0 | 98.0 | 116.1 | 0.101 | 1.00 |
| n-Butanol | 64.0 | >99.5 | 74.1 | 0.865 | 8.56 |
| Potassium hydroxide | 6.4 | 89.1 | 56.1 | 0.102 | 1.00 |
| (KOH) | | | | | |
| Potassium carbonate | 21.4 | >99.0 | 138.2 | 0.155 | 1.50 |
| (K₂CO₃) | | | | | |
| Dichlorodiethyl ether | 22.4 | >99.0 | 143.0 | 0.156 | 1.50 |
| (DCEE) | | | | | |
| Tetrabutylphosphonium | 1.5 | 40% | 276.4 | 0.002 | 0.02 |
| hydroxide (PTC) | | | | | |

### Procedure

DAH (12 g, 1 eq), n-butanol (64 g), powdered KOH (6.4 g, 1 eq), powdered K₂CO₃ (21.4 g, 1.5 eq) and tetrabutylphosphonium hydroxide (1.5 g at 40%) were charged in sequence to an Easymax 102 reactor. The suspension was heated to a gentle reflux (over 15 minutes) and DCEE (22.4 g, 1.5 eq) was fed sub-surface over 4 hours via syringe pump while water was azeotropically removed from the system. Azeotropic distillation continued post feed until the reaction was completed. The reaction was sampled periodically over a 12-hour period.

After reaction completion, the inorganic solids were removed via filtration at room temperature followed by a n-butanol wash (30 g) at room temperature. The combined filtrate (87 g) and wash (32 g) gave a solution yield of 55%.

### Example 9

TABLE 9

| | Weight | Purity | MW | Moles | Mole |
|---|---|---|---|---|---|
| Materials | (g) | (%) | (g/mol) | (moles) | Ratio |
| Diacetylhydrazine (DAH) | 52.9 | 98.0 | 116.1 | 0.447 | 1.00 |
| n-Butanol | 192.0 | >99.5 | 74.1 | 2.591 | 5.80 |
| Potassium carbonate | 154.4 | >99.0 | 138.2 | 1.117 | 2.50 |
| (K₂CO₃) | | | | | |
| Dichlorodiethyl ether | 100.8 | >99.0 | 143.0 | 0.705 | 1.54 |
| (DCEE) | | | | | |
| Tetrabutylphosphonium | 6.75 | 40% | 276.4 | 0.01 | 0.02 |
| hydroxide (PTC) | | | | | |

### Procedure

DAH (52.9 g, 1 eq), n-butanol (192 g), tetrabutylphosphonium hydroxide (6.75 g at 40%), powdered K₂CO₃ (154.4 g, 2.5 eq) were charged to an Easymax 402 reactor. After the suspension was heated to gentle reflux over 15 minutes, DCEE (100.8 g, 1.5 eq) was fed sub-surface over 4 hours via syringe pump and the reaction was held at reflux for additional 8 hours.

After reaction completion, the inorganic solids were removed via filtration at 80° C., which was followed by a hot n-butanol wash (150 g) at 80° C. The combined filtrate (250 g) and wash (155.8 g) were concentrated under reduced pressure at 60-65° C. to render a concentrate (92.6 g) containing about 55% of DAODA.

To crystallize DAODA, the concentrate was cooled slowly over 30 min to −5° C. with mixing. Product isolation through filtration, a cold n-butanol wash (34 g) at −5° C., and drying under vacuum at 60° C. afforded DAODA (37.5 g) in a 45% yield with a purity of 99%. The mother liquor (41.5 g), containing DAODA (6.1 g, 7.3% yield) and n-butanol wash (36.6 g) containing DAODA (3.4 g, 4% yield) were main sources of product loss.

### Example 10

TABLE 10

| | Weight | Purity | MW | Moles | Mole |
|---|---|---|---|---|---|
| Materials | (g) | (%) | (g/mol) | (moles) | Ratio |
| Diacetylhydrazine (DAH) | 18.0 | 98.0 | 116.1 | 0.152 | 1.00 |
| n-Butanol | 55.0 | >99.5 | 74.1 | 0.742 | 4.89 |
| Crystallization mother | 15.0 | / | / | / | / |
| liquor | | | | | |
| Potassium carbonate | 53.6 | >99.0 | 138.2 | 0.388 | 2.55 |
| (K₂CO₃) | | | | | |
| Dichlorodiethyl ether | 33.6 | >99.0 | 143.0 | 0.235 | 1.55 |
| (DCEE) | | | | | |

### Procedure

DAH (18 g, 1 eq), n-butanol (55 g), crystallization mother liquor (15 g, from Example 9), powdered K₂CO₃ (53.6 g, 2.5 eq) were charged to an Easymax 102 reactor. After the suspension was heated to gentle reflux over 15 minutes, DCEE (33.6 g, 1.5 eq) was fed sub-surface over 4 hours via syringe pump while maintaining the reaction temperature at gentle reflux. The reaction was held under reflux for additional 8 hours. The GC chemical yield for DAODA was 56%.

To enable a better determination reaction performance, DAODA and DCEE in the crystallization mother liquor were subtracted from the direct quantification in the reaction mass.

### Example 11

TABLE 11

| | Weight | Purity | MW | Moles | Mole |
|---|---|---|---|---|---|
| Materials | (g) | (%) | (g/mol) | (moles) | Ratio |
| Diacetylhydrazine (DAH) | 12.0 | 98.0 | 116.1 | 0.101 | 1.00 |
| 2-Methoxyethanol | 58.0 | 99.0 | 76.1 | 0.755 | 7.47 |
| Potassium carbonate | 37.5 | >99.0 | 138.2 | 0.271 | 2.55 |
| (K₂CO₃) | | | | | |
| Dichlorodiethyl ether | 22.4 | >99.0 | 143.0 | 0.156 | 1.50 |
| (DCEE) | | | | | |

### Procedure

DAH (12 g, 1 eq), 2-methoxyethanol (58 g) and powdered K₂CO₃ (37.5 g, 2.55 eq) were charged in sequence to an Easymax 102 reactor. The suspension was heated to a gentle reflux over 15 minutes and DCEE (22.4 g, 1.5 eq) was fed sub-surface over 4 hours via syringe pump. The reaction was held under reflux for additional 8 hours. The reaction was sampled periodically over a 12-hour period.

After reaction completion, the inorganic solids were removed via filtration at room temperature followed by a 2-methoxyethanol wash (25 g) at room temperature. The combined filtrate (95.4 g) gave a solution yield of 58%.

### Example 12

TABLE 12

| | Weight | Purity | MW | Moles | Mole |
|---|---|---|---|---|---|
| Materials | (g) | (%) | (g/mol) | (moles) | Ratio |
| Diacetylhydrazine (DAH) | 18.0 | 98.0 | 116.1 | 0.152 | 1.00 |
| 2-Methoxyethanol | 64.0 | 99.0 | 76.1 | 0.833 | 5.48 |
| Potassium carbonate | 42.0 | >99.0 | 138.2 | 0.304 | 2.00 |
| (K₂CO₃) | | | | | |
| Dichlorodiethyl ether | 33.6 | >99.0 | 143.0 | 0.233 | 1.53 |
| (DCEE) | | | | | |

### Procedure

DAH (18 g, 1 eq), 2-methoxyethanol (64 g) and powdered K₂CO₃ (42.0 g, 2.0 eq) were charged in sequence to an Easymax 102 reactor. The suspension was heated to a gentle reflux over 15 minutes and DCEE (33.6 g, 1.53 eq) was fed sub-surface over 4 hours via syringe pump. The reaction was held under reflux for additional 8 hours. The reaction was sampled periodically over a 12-hour period.

After reaction completion, the inorganic solids were removed via filtration at room temperature followed by a 2-methoxyethanol wash (40 g) at room temperature. The combined filtrate (149.9 g) gave a solution yield of 54.8%.

It can therefore be seen that the claimed invention provides a reaction that is efficient, low-cost and safer than that known in the prior art.

The invention is defined by the claims.

1. A process for the preparation of a compound of formula (I): 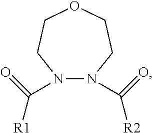
comprising the reaction of:
R¹—C(O)—NH—NH—C(O)—R², with R³—CH₂—CH₂—O—CH₂—CH₂—R⁴,
in the presence of an alcoholic solvent;
wherein R¹ and R² are independently selected from straight or branched chain alkyl groups, or are bonded to form a 4, 5, or 6-membered heterocycle, and
wherein R³ and R⁴ are independently selected from a halide or a sulphate.

2. A process according to claim 1, wherein R¹ and R² are i) methyl; or ii) are bonded to form a 5-membered heterocycle.

3. A process according to claim 1, wherein R³ and R⁴ are independently selected from methylsulfonyl, chlorine and/or bromine, preferably chlorine.

4. A process according to claim 1, wherein the alcoholic solvent is selected from a monohydric alcohol.

5. A process according to claim 4, wherein the solvent is selected from butanol, pentanol, or 2-methoxyethanol, preferably butanol.

6. A process according to claim 1, where the process is carried out in the presence of a phase-transfer catalyst (PTC).

7. A process according to claim 6, wherein the PTC is a nucleophilic catalyst, an ammonium catalyst or phosphonium catalyst.

8. A process according to claim 6, wherein the PTC is selected from 1,4-diazabicyclo[2.2.2]octane (DABCO), quinuclidine, Trimethylamine hydrochloride (TMA HCl), Tetrabutylphosphonium chloride (TBPCl), Tetrabutylphosphonium hydroxide (TBPOH) and Tributyltetradecylphosphonium chloride (TBTDPCl), or mixtures thereof.

9. A process according to claim 1, where the process is carried out in the presence of a base.

10. A process according to claim 9, wherein the process is carried out in the presence of a base and a co-base.

11. A process according to claim 10, wherein the ratio of base to co-base is from 10:1 to 1:2, preferably from 5:1 to 1:1.

12. A process according to claim 9, wherein the base is selected from potassium carbonate, sodium carbonate and caesium carbonate, or mixtures thereof.

13. A process according to any of claims 10 to 12, wherein the co-base is selected from potassium hydroxide, potassium n-butoxide, sodium hydroxide, caesium hydroxide, and mixtures thereof.

14. A process according to claim 1, any of the preceding claims, wherein the reaction is carried out at at least one of the solvent reflux temperature or 110 to 125° C.

15. A process according to claim 1, comprising the removal of water via azeotropic distillation.

16. A process according to claim 1, comprising a salt filtration step.

17. A process according to claim 1, comprising a solvent distillation step, preferably only one solvent distillation step.

18. A process according to claim 1, comprising a product crystallisation step.

19. A process for preparing pinoxaden comprising a process as defined in claim 1.

20. A compound of formula (I) produced by a process as defined in claim 1.
